(19) United States
(12) Patent Application Publication
(10) Pub. No.: US 2006/0263787 A1
(43) Pub. Date: Nov. 23, 2006

(54) Immunoglobulin-like variable chain binding polypeptides and methods of use

(76) Inventors: Glen A. Evans, San Marcos, CA (US); Katya McLane, Vista, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/126,105

(22) Filed: May 10, 2005

Related U.S. Application Data
(60) Provisional application No. 60/569,920, filed on May 10, 2004.

(51) Int. Cl. C12Q 1/68; G01N 33/53; C07K 16/24; C07K 16/26; C07K 16/30

(52) U.S. Cl. 435/6; 435/7.1; 530/388.23; 530/388.24; 530/388.25

(57) ABSTRACT

The invention provides an isolated diverse population of V_{H}-like binding polypeptides. Each binding polypeptide within the population comprising an unascertained combination of an immunoglobulin V_{H} region exon encoded polypeptide, a J_{H} region exon encoded polypeptide and a D region exon encoded polypeptide, wherein the V_{H}, D and J_{H} region exon encoded polypeptides are joined in a single polypeptide forming an immunoglobulin V_{H}-like binding polypeptide, or a functional fragment thereof.

### CROSS-REFERENCE TO RELATED APPLICATION

This application claims the benefit of U.S. Provisional Application No. 60/569,920, filed 10 May 2004, entitled, “Immunoglobulin-Like Variable Chain Binding Polypeptides and Methods of Us,” the entire contents of which is incorporated herein by reference.

This invention relates generally to the methods of producing populations of binding polypeptides and, more specifically to immunoglobulin-like binding polypeptides that recognize a particular ligand.

The war on fatal, debilitating and chronic diseases has entered the twenty-first century. Recent years have shown tremendous progress in the understanding of the development and progression of certain diseases. However, there has been only marginal decreases in death rates from most types of fatal diseases and the treatment of many debilitating and chronic diseases still has major hurdles to overcome before cures can be expected. For example, cancer remains a major fatal disease. Standard chemotherapy and radiation therapy generally involve treatment with therapeutic agents that impact not only the diseased cells but also other proliferative cells of the body, often leading to debilitating side effects. Therefore, it remains desirable to identify therapeutic agents with a higher degree of specificity for the carcinogenic lesion. Similarly, therapeutic agents with greater specificity also are desirable to both increase efficacy and lower undesirable side effects.

The discovery of monoclonal antibodies (mAbs) in the 1970's provided great hope for the reality of creating therapeutic molecules with high specificity. Antibodies that bind to diseased cell antigens would provide specific targeting agents for therapy. While the development of monoclonal antibodies has provided a valuable diagnostic reagent, certain limitations restrict their use as therapeutic entities.

Because mAbs are usually developed in non-human species, one limitation encountered when attempts are made to use them as therapeutic agents is that they elicit an immune response in human patients. Chimeric antibodies join the variable region binding domain of the non-human species to a human antibody constant region. However, the chimeric antibody often remains immunogenic and it is therefore necessary to further modify the variable region.

One modification is the grafting of complementarity-determining regions, (CDRs) which impart antigen binding onto a human antibody variable framework. This approach is imperfect because CDR grafting often diminishes the binding activity of the resulting humanized mAb. Other modifications that have been employed to reduce immunogenicity of a chimeric, humanized or non-human antibody include veneering and resurfacing of the antibody solvent exposed residues to remove T- and B-cell antigenic epitopes.

Attempts to regain binding activity or to remove antigenic epitopes on humanized or other types of modified antibodies require laborious, step-wise procedures which have been pursued essentially by a trial and error type of approach. For example, one difficulty in regaining binding affinity is because it is difficult to predict which framework residues serve an important role in maintaining antigen binding affinity and specificity.

Combinatorial methods have been applied to restore binding affinity, however, these methods require sequential rounds of mutagenesis and affinity selection that can both be laborious and unpredictable. Consequently, while antibody humanization methods that rely on structural and homology data are used, the complexity that arises from the large number of framework residues potentially involved in binding activity has hindered rapid success.

Thus, there exists a need for therapeutic binding molecules that exhibit specific binding characteristics as monoclonal antibodies and can be efficiently produced. The present invention satisfies this need and provides related advantages as well.

### SUMMARY OF THE INVENTION

The invention provides an isolated diverse population of V_{H}-like binding polypeptides. Each binding polypeptide within the population comprising an unascertained combination of an immunoglobulin V_{H} region exon encoded polypeptide, a J_{H} region exon encoded polypeptide and a D region exon encoded polypeptide, wherein the V_{H}, D and J_{H} region exon encoded polypeptides are joined in a single polypeptide forming an immunoglobulin V_{H}-like binding polypeptide, or a functional fragment thereof. The invention also provides an isolated diverse population of V_{L}-like binding polypeptides, each binding polypeptide within the population comprising an unascertained combination of an immunoglobulin V_{L} region exon encoded polypeptide and a J_{L} region exon encoded polypeptide, wherein the V_{L} region exon encoded polypeptide and the J region exon encoded polypeptide are joined in a single polypeptide forming an immunoglobulin V_{L}-like binding polypeptide, or a functional fragment thereof. Further, an isolated diverse population of F_{V}-like binding polypeptides. Each binding polypeptide within the population F_{V}-like binding polypeptides comprising an unascertained combination of a V_{H}-like binding polypeptide and a V_{L}-like binding polypeptide, each of the V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of the V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein the V_{H}-like and the V_{L}-like binding polypeptides associate to form an immunoglobulin F_{V}-like binding polypeptide.

### BRIEF DESCRIPTION OF THE DRAWINGS

FIG. 1 shows a schematic diagram of the topography of an immunoglobulin and a immunoglobulin functional Fab fragment.

FIG. 2 shows J_{H} region exon encoded polypeptide sequences for exons J_{H1}-J_{H6}.

FIG. 3 shows D region exon encoded polypeptide sequences for exons D1-D7.

FIG. 4 shows the diversity that can be generated by combining V, D and J exons.

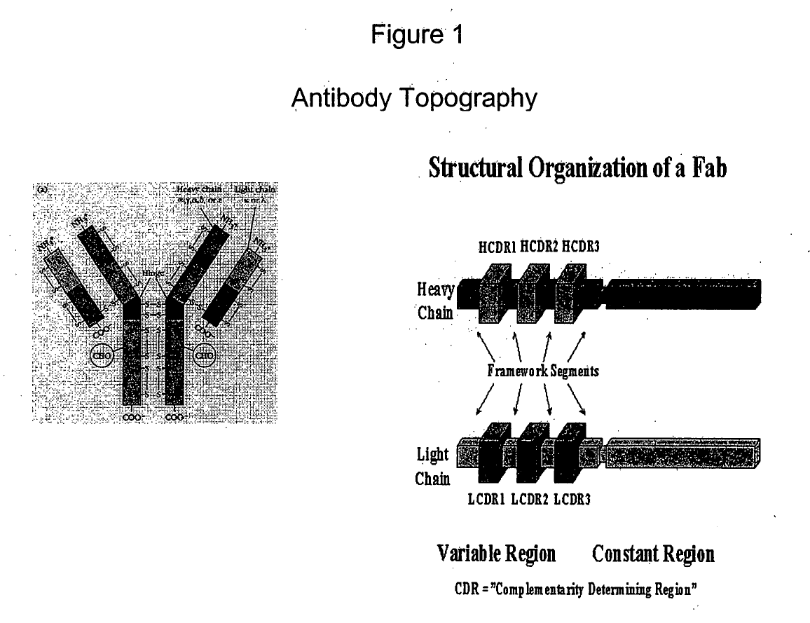

### DETAILED DESCRIPTION OF THE INVENTION

The invention is directed to populations of immunoglobulin-like binding polypeptides that contain members exhibiting a wide range of binding specificities. The populations can be screened for specific binding activity to a predetermined ligand. Immunoglobulin-like binding polypeptides of the invention include V_{H}-like, V_{L}-like and F_{V}-like binding polypeptides. F_{V}-like binding polypeptides result from assembly of a V_{H}-like and a V_{L}-like binding polypeptide. These binding polypeptides as well as functional fragments exhibiting binding specificity to a ligand also are included as an immunoglobulin-like binding polypeptide of the invention. The immunoglobulin-like binding polypeptides of the invention mimic the structure of an authentic immunoglobulin polypeptide or binding fragment thereof. Therefore, the immunogloulin-like binding polypeptides of the invention exhibit beneficial characteristics of authentic immunoglobulins such as molecular stability and specific binding affinity to a target ligand. Additionally, the methods of producing the immunoglobulin-like binding polypeptides of the invention allow the efficient use of human nucleic acid encoding sequences so that their immunogenecity when used as a human therapeutic is negligible. Immunoglobulin-like binding polypeptides of the invention can be rapidly and efficiently generated to increase the availability, specificity or efficacy of useful therapeutics for human diseases.

In one embodiment, the invention is directed to an isolated diverse population of V_{H}-like binding polypeptides having unascertained combinations of exon encoded polypeptide sequences corresponding to variable (V), diversity (D) and joining (J) regions of an immunoglobulin heavy chain variable region. The binding polypeptides can be produced by nucleic acid synthesis and joining of any or all possible combinations of exons encoding these regions and translation into polypeptides. Exon encoded D and J regions are joined into a polypeptide corresponding to the third complementarity determining region (CDR) of an immunoglobulin. Unascertained combinations of such a J_{H} region polypeptides with a V_{H} region exon encoded polypeptide can be compiled into a diverse population consisting of some or all combinations of CDRs 1 and 2 encoded in V_{H} exons and CDR3 encoded in D and J exons giving rise to J_{H} polypeptides. Resulting polypeptides consist of a V_{H}-like polypeptide having similar primary, secondary and tertiary structure and similar function to a V_{H} immunoglobulin chain.

In another embodiment, the invention is directed to an isolated diverse population of V_{L}-like binding polypeptides having unascertained combinations of exon encoded polypeptide sequences corresponding to variable (V) and joining (J) regions of an immunoglobulin light chain variable region. Nucleic acid synthesis and joining of any or all possible combinations of exons encoding these regions with translation into polypeptides similarly can be employed to produce V_{L}-like binding polypeptides. Unascertained combinations of a J_{L} region exon encoded polypeptide with a V_{L} region exon encoded polypeptide can be compiled into a diverse population consisting of some or all combinations of CDRs 1 and 2 encoded in V_{L} exons and CDR3 encoded in J_{L} exons resulting in a V_{L}-like polypeptide having similar primary, secondary and tertiary structure and similar function to a V_{L} immunoglobulin chain.

In a further embodiment, the invention is directed to a diverse population of F_{V}-like binding polypeptides having unascertained combinations of V_{H}-like and V_{L}-like binding polypeptides. Populations of F_{V}-like binding polypeptides as well as V_{H}-like or V_{L}-like binding polypeptides can be screened and one or more molecules identified for specific binding activity to a target ligand. Functional fragments such as F_{d}-like binding polypeptides also can be produced and isolated for target specific binding activity.

As used herein, the term “isolated” when used in connection with a population of binding polypeptides or encoding nucleic acids is intended to mean that the population of molecules is in a form outside of a mammalian organism. Accordingly, the term refers to a population of binding polypeptides or encoding nucleic acids that is relatively free from organismic components such as tissues and other organ systems. Isolated populations of V_{H}-like binding polypeptides, V_{L}-like binding polypeptides or F_{V}-like binding polypeptides include, for example, pure population forms that are substantially free from other polypeptide or nucleic acid species or from organismic or cellular contaminants. Isolated populations also include, for example, populations existing in a culture medium, cell extract or cell fractionation form derived from binding polypeptide-producing cells or populations encoded in a cell population for propagation, expression or further manipulations. Accordingly, the term refers to substantially pure populations as well as populations that can be found in an environment distinct from a naturally occurring immunoglobulin repertoire.

The term “substantially pure” as used herein with reference to an individual polypeptide or encoding nucleic acid is intended to mean that the molecule is in a form that is relatively free from cellular components or other contaminants that are not the desired molecule. A substantially pure polypeptide or nucleic acid also can be sufficiently homogeneous so as to resolved by physical or biophysical separation techniques such as an electrophoretic band, a chromatographic peak or a sequence profile consistent with a predominant species. Accordingly, a substantially pure polypeptide or nucleic acid of the invention refers to a polypeptide or nucleic acid that is enriched compared to lipids, other polypeptide or nucleic acid species or other cellular material associated with an immunoglobulin in its natural state.

As used herein, the term “diverse” when used in reference to a population is intended to refer to a population having a multiplicity of different constituent members. Therefore, the term “diverse” refers to a population exhibiting diversity or variety between members of the population. Variety of constituent members can result from differences in nucleotide or amino acid sequences, differences in activities or differences in structure. Differences in sequences can be due to, for example, overall differences between complete or whole sequences or differences between regions of two or more compared sequences. Such regions can reference activity or structural domains, for example. Differences in activities when used in reference to binding activity can include, for example, dissimilarities in binding affinity, avidity, valancy, dissociation rate or association rate. Differences in structure can include, for example, dissimilarity in two- or three-dimensional structure as well as dissimilarities in tertiary or higher order structures. Variety of constituent members also can occur from differences in, for example, catalytic activity resulting from dissimilarities such as velocity, maximum velocity and turn-over rate. Accordingly, a diverse population refers to a population composed of various differences in sequence, activity or structure.

As used herein, the term “population” is intended to mean a collection of two or more molecules. A population can contain a few or a large number of different molecules, varying from as small as 2 molecules to as large as 10¹³ or more molecules. Therefore, a population can range in size from 2 to 10, 10 to 100, 100 to 10³, 10³ to 10⁵, 10⁵ to 10⁸, 10⁸ to 10¹⁰ or 10¹⁰ to 10¹³ molecules. The molecules making up a population can be nucleic acid molecules such as a DNA or RNA. Such types of nucleic acids can include, for example, genomic DNA, hnRNA, mRNA, rRNA, cDNA or chemically synthesized DNA or RNA. Additionally, the molecules making up a population of the invention also can be polypeptide molecules including variant or modified polypeptide or polypeptide containing one or more amino acid analogs. Similarly, the molecules making up a population can be polypeptide-like molecules, referred to herein as peptidomimetics, which mimic the activity of an amino acid or polypeptide; or a polypeptide such as an enzyme or a fragment thereof. Moreover, a population can be diverse or redundant depending on the intent and needs of the user. Those skilled in the art will know the size and diversity of a population suitable for a particular application.

As used herein, the term “immunoglobulin” is intended to mean a vertebrate antibody serum protein consisting of heavy and light chains usually linked by disulfide bonds and able to bind a specific molecular target. Heavy and light immunoglobulin chains are further composed of variable and constant region domains where the variable regions confer target binding activity. Immunoglobulins are produced by mammalian B cells and constitute one component of an organisms humoral immune system. The structure and function of an immunoglobulin molecule is well known to those skilled in the art and can be found described in, for example, Paul, W. E., Fundamental Immunology, Lippincott Williams & Wilkins Publishers, Fifth Ed., Baltimore, Md. (2003); Lodish et al., Molecular Cell Biology, Scientific American Books, Third Ed., New York, N.Y., (1996); Meyers, R. A., Molecular Biology and Biotechnology: A Comprehensive Desk Reference, VCH Publishers, New York, N.Y., (1995); Borrebaeck (Ed.), Antibody Engineering (Second edition) New York: Oxford University Press (1995); Borrebaeck (Ed.), Antibody Engineering (Second Ed.) New York: Oxford University Press (1995), and Harlow and Lane, Antibodies: A Laboratory Manual, Cold Spring Harbor Laboratory Press, NY (1988).

A variable region of an immunoglobulin heavy or light chain refers to the amino terminal portion of each chain which participates in antigen binding. Each variable region chain is about 100 to 110 amino acids in length and is structurally and functionally separated into domains. Complementarity determining regions (CDR), when referenced by structure, or hypervariable regions when referenced by sequence variability, correspond to one category of domains within a variable region. Framework (Fw) regions correspond to another category of domains. Immunoglobulin variable regions or V regions have an organization structure consisting of three CDRs interspersed by Fw regions. Immunoglobulins contain an art-recognized β-sandwich structural motif that folds the CDR domains in three-dimensional space to form an antigen binding pocket. The structure and function of immunoglobulin variable regions are well known in the art and can be found described in, for example, Paul, W. E., supra; Lodish et al., supra; Meyers, R. A., supra; Borrebaeck (Ed.), supra, and Harlow and Lane, supra. A binding polypeptide that mimics a V_{H}-like or a V_{L}-like region will exhibit about the same binding activity toward an antigen as a V_{H} or V_{L} region and contain CDR and Fw regions. The CDR or Fw regions can be structurally similar to variable region CDR or Fw region domains of an immunoglobulin. V_{H}-like or V_{L}-like regions will contain CDRs folded into a three-dimensional antigen binding pocket.

CDRs of an immunoglobulin variable region correspond to a region containing one of three hypervariable loops (H1, H2 or H3) within the non-framework region of an immunoglobulin V_{H} β-sheet framework, or a region containing one of three hypervariable loops (L1, L2 or L3) within the non-framework region an immunoglobulin V_{L} β-sheet framework. CDR regions are well known to those skilled in the art and have been defined by, for example, Kabat as the regions of most hypervariablity within the immunoglobulin variable domains (see, for example, Kabat et al., J. Biol. Chem. 252:6609-16 (1977), and Kabat, Adv. Prot. Chem. 32:1-75 (1978)). CDR region sequences also have been defined structurally by Chothia as those residues that are not part of the conserved β-sheet framework, and thus are able to adapt different conformations (see, for example, Chothia and Lesk, J. Mol. Biol. 196:901-17 (1987)). Both terminologies are well known to those skilled in the art. The positions of CDRs within a canonical immunoglobulin variable domain have been determined by comparison of numerous structures (see, for example, Al-Lazikani et al., J. Mol. Biol. 273:927-48 (1997); Morea et al., Methods 20:267079 (2000)). Because the number of residues within a loop varies in different immunoglobulins, additional loop residues relative to the canonical positions are conventionally numbered with a, b, c, and so forth next to the residue number in the canonical variable domain numbering scheme (Al-Lazikani et al., supra (1997)). Such nomenclature is similarly well known to those skilled in the art.

For example, CDRs defined according to either Kabat (hypervariable) or Chothia (structural) designations, are set forth below in Table 1.

TABLE 1
CDR Definitions

| CDR | Kabat¹ | Chothia² | Loop Location |
|---|---|---|---|
| VH CDR1 | 31-35 | 26-32 | linking B and C strands |
| VH CDR2 | 50-65 | 53-55 | linking C′ and C″ strands |
| VH CDR3 | 95-102 | 96-101 | linking F and G strands |
| VL CDR1 | 24-34 | 26-32 | linking B and C strands |
| VL CDR2 | 50-56 | 50-52 | linking C′ and C″ strands |
| VL CDR3 | 89-97 | 91-96 | linking F and G strands |

¹Residue numbering follows the nomenclature of Kabat et al., supra.
²Residue numbering follows the nomenclature of Chothia et al., supra.

Immunoglobulin variable region framework or framework domain corresponds to the portion or portions on an immunoglobulin heavy or light chain variable region other than the CDRs. An immunoglobulin variable framework will contain about four Fw domains that correspond to the amino acids that flank or intervene between the three CDR region sequences. For example, Fw region 1 corresponds to amino acid residues amino terminal to CDR1. Framework region 2 corresponds to the amino acid residues separating CDRs 1 and 2. Similarly, Fw region 3 corresponds to the amino acid residues separating CDRs 2 and 3 while Fw region 4 corresponds to the amino acid residues carboxy terminal to CDR3. Immunoglobulin variable region frameworks are well known to those skilled in the art and can be found described in, for example, Paul, W. E., supra, as well as in Lodish et al., supra; Meyers, R. A., supra; Harlow and Lane, supra, Kabat et al., supra, (1977); Kabat, supra, (1978); Chothia and Lesk, supra; Al-Lazikani et al., supra, and Morea et al., supra.

Immunoglobulin J and D regions correspond to portions of CDR3 that are defined in the art by an immunoglobulin exon encoding sequence. As with other variable region sequences, J and D region sequences are combined into a linear contiguous sequence with adjacent variable region sequences by somatic recombination. D and J region sequences can be found as part of CDR3 region sequences in immunoglobulin V_{H} domains. J region sequences can be found as part of CDR3 region sequences in immunoglobulin V_{L} domains. There are about 25 different functional D region exons and about 6 different functional J region exons where one D region exon and one J region exon each encode a different portion of a V_{H} CDR3. J region exons for V_{L} domains are selected from a family of about five J_{κ} and about four J_{λ} functional exon sequences. Descriptions of D region exon encoded sequences when made in context of both V_{H} and V_{L} chains will be denoted herein by use of parenthesis such as (D) to represent that a D region exon encoded polypeptide sequence is present only with respect to the V_{H} chain binding polypeptide or encoding nucleic acid sequence.

The term “exon encoded polypeptide” as it is used herein is intended to refer to the amino acid sequence encoded by a referenced exon. Accordingly, a V_{H} exon encoded polypeptide refers to the amino acid portion encoded by a V_{H} chain exon. Similarly, a D region exon encoded polypeptide or a J region exon encoded polypeptide refer to the amino acid portion encoded by a D or J region exon, respectively. Variable region exons, D region exons and J region exons that combine to encode a functional immunoglobulin V_{H} or V_{L} chain domain as well as their genomic arrangement and mechanism of recombination are well known in the art. The genomic structure, encoding nucleic acid sequence, amino acid sequence and method of recombination can be found described in, for example, Paul, W. E., Fundamental Immunology, Lippincott Williams & Wilkins Publishers, Fifth Ed., Baltimore, Md. (2003); as well as in Lodish et al., supra; Meyers, R. A., supra; Harlow and Lane, supra, Kabat et al., supra, (1977); Kabat, supra, (1978); Chothia and Lesk, supra; Al-Lazikani et al., supra, and Morea et al., supra.

As used herein, the term “V_{H}-like” or “heavy chain variable region-like” when used in reference to a binding polypeptide is intended to mean a polypeptide that structurally mimics an immunoglobulin heavy chain variable region polypeptide. Similarly, the term “V_{L}-like” or “light chain variable region-like” when used in reference to a binding polypeptide is intended to mean a polypeptide that structurally mimics an immunoglobulin light chain variable region polypeptide. Structural mimicry for V_{H}-like or V_{L}-like polypeptides can be at the primary, secondary or tertiary structural level. A V_{H}-like or V_{L}-like binding polypeptide of the invention that mimics a heavy chain variable (V_{H}) region or a light chain variable V_{L} region will imitate or copy a primary, secondary or tertiary structure of an immunoglobulin V_{H} or V_{L} polypeptide. Accordingly, a V_{H}-like or a V_{L}-like binding polypeptide will consist, for example, of about three CDR domains interspersed by Fw region sequences. An exemplary structure of a V_{H}-like or a V_{L}-like binding polypeptide can be, for example, amino acid sequences conferring the functions of NH₂-Fw1-CDR1-Fw2-CDR2-Fw3-CDR3-Fw4-CO₂H.

As used herein, the term “F_{V}-like” or “variable region-like” when used in reference to a binding polypeptide is intended to mean a multimeric polypeptide that structurally mimics an immunoglobulin variable region domain and exhibits binding affinity to a ligand. Components of a F_{V}-like binding polypeptide will include at least one V_{H}-like and at least one V_{L}-like polypeptide associated to form an antigen binding pocket. The antigen binding pocket within such a tertiary F_{V}-like structure being composed of V_{H}-like CDR sequences and V_{L}-like CDR sequences in relative proximity to each other in three-dimensional space so as to form a binding pocket. The term is similarly intended to include functional binding equivalents corresponding to immunoglobulin variable region fragments such as Fab, F(ab)₂, single chain F_{V} (scF_{V}) and the like.

As used herein, the term “functional fragment” when used in reference to a V_{H}-like, V_{L}-like or F_{V}-like binding polypeptide, or immunoglobulin-like binding polypeptide, of the invention is intended to mean a portion of an immunoglobulin-like binding polypeptide which retains at least about the same ligand binding activity compared to the parent or intact immunoglobulin-like binding polypeptide. Binding activity can be retained, for example, where the primary, secondary or tertiary structure of the V_{H}-like or V_{L}-like polypeptides is substantially retained. Such functional fragments can include, for example, truncated, deleted or substituted amino acid residues of the parent or intact immunoglobulin-like binding polypeptide so long as it retains about the same binding activity as the reference immunoglobulin-like binding polypeptide. A specific example of a functional fragment of an F_{V}-like binding polypeptide of the invention is an F_{d}-like binding polypeptide, which corresponds to an immunoglobulin F_{d} dimeric fragment consisting of V_{H} and V_{L} chain portions containing all three CDRs but truncated above the cystine residues that function in interchain disulfide bonding. An F_{d}-like binding polypeptide consists of, for example, the V_{H} and V_{L} chain portions substituted with V_{H}-like and V_{L}-like chains, respectively. Functional fragments of V_{H}-like, V_{L}-like or F_{V}-like binding polypeptides of the invention include, for example, protions of a V_{H}-like, V_{L}-like or F_{V}-like binding polypeptide containing one or more CDRs conferring binding contacts with a target ligand, F_{d} and the like. Functional fragments of immunoglobulins are well known to those skilled in the art. Accordingly, the use of these terms in describing functional fragments of the immunoglobulin-like binding polypeptides of the invention is intended to correspond to the definitions of immunoglobulin functional fragments well known to those skilled in the art. Such terms are described in, for example, Harlow and Lane, supra; Myers, R. A., supra; Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990).

Immunoglobulin-like polypeptides of the invention and functional fragments thereof are intended to include polypeptides having minor modifications of a specified amino acid sequence but which exhibits at least about the same ligand binding activity as the referenced V_{H}-like, V_{L}-like or F_{V}-like binding polypeptide. Minor modifications of a polypeptide having at least about the same ligand binding activity as the referenced polypeptide include, for example, conservative substitutions of naturally occurring amino acids as well as structural alterations which incorporate non-naturally occurring amino aids, amino acid analogs and functional mimetics.

For example, a Lysine (Lys) is considered to be a conservative substitution for the amino acid Arginine (Arg). Other conservative amino acid substitutions and functional equivalents are well known in the art and can be found described in, for example, Lehninger, Principles of Biochemistry, Nelson and Cox, Third Ed., Worth Publishers, New York (2000), and in Stryer, Biochemistry, Fourth Ed. W.H. Freeman and Company, New York (1995). Similarly, analog or mimetic structures substituting positive or negative charged or neutral amino acids, with organic structures having similar charged and special arrangements also is considered a functional equivalent of a referenced amino acid sequence so long as the polypeptide analog or mimetic exhibits at least about the same ligand binding activity as the referenced polypeptide. Given the teachings and guidance provided herein, those skilled in the art will know, or can determine, which mimetic structures will function as an equivalent of an immunoglobulin-like binding polypeptide or as a domain or amino acid residue thereof.

As used herein, the term “unascertained” when used in reference to polypeptide or polypeptide domain combinations is intended to mean that the order or constituent polypeptides of the resulting combination are not determined, resolved or settled in advance of the events forming the combination. The term is intended to relieve any imposition of knowledge or predisposition on the order or constituent members of the referenced combinations. Therefore, an unascertained combination refers to polypeptide or domain combinations that lack a predetermined certainty with reference to the resulting combinations in advance of the pairings.

As used herein, the term “specific” when used in reference to a polypeptide binding activity is intended to mean that the polypeptide exhibits discriminating or preferential binding activity toward a target ligand compared to a non-target ligand. An immunoglobulin-like binding polypeptide exhibiting specific binding activity will distinguish or recognize a target ligand preferentially over non-target ligands, other polypeptides or macromolecules. Preferential binding can be due to specificity, affinity, avidity off rate, on rate or any combination thereof. Those skilled in the art will know, or can determine, preferential binding of a target ligand using binding methods well known to those skilled in the art.

As used herein, the term “polypeptide” is intended to mean two or more amino acids covalently bonded together. The size of a polypeptide of the invention can include short sequences from about two or more amino acid residues as well as large sequences consisting of fifty or a hundred or more residues as well as several hundred to a thousand or more amino acid residues. Accordingly, a polypeptide of the invention can be of any length greater than two amino acids and includes, for example, V_{H}-like, V_{L}-like and F_{V}-like polypeptides as well as functional fragments thereof. Generally, the covalent bond between the two or more amino acid residues is an amide bond. However, the amino acids can be joined together by various other means known to those skilled in the polypeptide and chemical arts. Therefore, the term “polypeptide” is intended to include molecules which contain, in whole or in part, non-amide linkages between amino acids, amino acid analogs and mimetics. Similarly, the term also includes cyclic polypeptide and other conformationally constrained structures.

As used herein, the term “nucleic acid” is intended to mean a single- or double-stranded DNA or RNA molecule. A nucleic acid molecule can be linear, circular or branched configuration, and can represent either the sense or antisense strand, or both, of a nucleic acid sequence. The term also is intended to include nucleic acid molecules of both synthetic and natural origin. A nucleic acid molecule of natural origin can be derived from any animal, such as a human, non-human primate, mouse, rat, rabbit, bovine porcine, ovine, canine, feline, or amphibian, or from a lower eukaryote such as Drosophila, C. elegans or yeast. A synthetic nucleic acid includes, for example, chemical and enzymatic synthesis. The term “nucleic acid” similarly is intended to include analogues of natural nucleotides which have similar functional properties as the referenced nucleic acid and which can be utilized in a manner similar to naturally occurring nucleotides and nucleosides.

As use herein, the term “coexpressing” or “coexpression” is intended to mean the expression of two or more molecules by the same cell. Coexpressed molecules can be polypeptides or encoding nucleic acids. Coexpression can be achieved by, for example, constitutive or inducible methods, including natural or recombinant means. Coexpression of two or more nucleic acids or polypeptides can occur, for example, simultaneously or sequentially and can be co-regulated or regulated independently. Various combinations of these and other modes well known to those skilled in the art can additionally be used depending on the number and intended use of the coexpressed molecules. The term is intended to include the coexpression of members originating from different populations in the same cell. For example, populations of molecules can be coexpressed where single or multiple different species from two or more populations are expressed in the same cell. A specific example includes the coexpression of V_{H}-like and V_{L}-like binding polypeptide populations where at least one member from each population is expressed together in the same cell to produce a library of cells coexpressing different species of F_{V}-like binding polypeptides. Populations which can be coexpressed can be as small as two different species within each population. Additionally, the number of molecules coexpressed from different populations also can be as large as, for example, 10⁵, 10⁶, 10⁷, 10⁸, 10⁹ or 10¹⁰ as well as 10¹³ or greater. Numerous different sized populations of nucleic acids or polypeptides between the above ranges and greater also can be coexpressed. Those skilled in the art know or can determine what modes of coexpression can be used to achieve a particular sized population of two or more different species of molecules.

The invention provides an isolated diverse population of V_{H}-like binding polypeptides. Each binding polypeptide within the population consists of an unascertained combination of an immunoglobulin V_{H} region exon encoded polypeptide, and a J_{H} region polypeptide consisting of an unascertained combination of a J_{H} region exon encoded polypeptide portion and a D region exon encoded polypeptide portion. The V_{H} region exon encoded polypeptide and the J_{H} region polypeptide are joined in a single polypeptide forming an immunoglobulin V_{H}-like binding polypeptide, or a functional fragment thereof.

The invention also provides an isolated diverse population of V_{L}-like binding polypeptides. Each binding polypeptide within the population consists of an unascertained combination of an immunoglobulin V_{L} region exon encoded polypeptide, and a J_{L} region exon encoded polypeptide portion. The V_{L} region exon encoded polypeptide and the J region exon encoded polypeptide are joined in a single polypeptide forming an immunoglobulin V_{L}-like binding polypeptide, or a functional fragment thereof.

The diversity of the immunoglobulin repertoire within an animal's immune system is enormous and the capacity of an immune system has appeared hard to saturate. The invention harnesses modular components of immunoglobulin molecules to mimic the in vivo diversity of an immune system immunoglobulin repertoire. Genomic nucleic acid sequences encoding portions of immunoglobulin binding domains are synthesized and combined in a fashion that imitates the results of somatic recombination to generate large populations of immunoglobulin-like binding polypeptides exhibiting diverse binding specificities. The synthesized immunoglobulin portions correspond to variable region exon nucleotide sequences. For the heavy chain variable region (V_{H}) these exons correspond to the amino terminal portion of V_{H}, the D region and the J region amino acid sequences. For the light chain variable region (V_{L}) these exons correspond to the amino terminal portion of V_{L} and the J_{L} region amino acid sequences. Synthesis and combination can occur at the nucleic acid level or at the polypeptide level. Use of exon encoding nucleotide sequences is amenable to recombinant methods for efficient generation large libraries of encoding nucleic acids or expressed polypeptides. Diversity of resultant populations can be modulated by varying the starting number of different exons for some or all of the exon encoded regions or by varying the length or sequence of exon encoded regions.

Immunoglobulin V region genes are encoded by families of V, (D) and J exons corresponding to different portions of the immunoglobulin V region. During development antibody producing B lymphocytes rearrange the genomic DNA to combine one exon from each of these families to generate a V_{H} encoding gene and one V and J exon from each family to generate a V_{L} encoding gene. The process of generating a contiguous V_{H} encoding exon from exon families encoding portions of the complete V_{H} gene is termed somatic recombination and is an in vivo source for immunoglobulin binding diversity.

There are two sources of immunoglobulin sequence diversity, and concomitant binding diversity, arising from in vivo somatic recombination. Once source is termed “germline diversity” which is generated through semi-random combinations of V, (D) and J region exons into a contiguous and complete V region gene. Briefly, the human V_{H} region is encoded by a family of about 51 different functional heavy chain V_{H} exons. Recombination of one of these V_{H} exons can occur with any of about 25 different D region exons and any of about 6 different J region exons. Similarly, the human V_{L} region is encoded by a family of about 70 different functional V_{L} region exons. The V_{L} exons consist of about 40 different V_{κ} exons and about 30 different V_{λ} exons. Recombination of a V_{κ} exon can occur with any of about 5 different J_{κ} exons and recombination of a V_{λ} exon can occur with any of about 4 different J_{λ} exons.

The second source of in vivo immunoglobulin sequence diversity is termed “junctional diversity,” which refers to the diversity generated through imprecise joining of V, (D) and J exon segments. In this regard, during somatic recombination, a variable number of nucleotides are deleted and additional nucleotides are incorporated randomly by template-independent deoxynucleotidyl transferase (terminal transferase). Both junctional and germline diversity are processes undergone by individual immunoglobulin producing B lymphocytes during development. Random and imprecise joining of different V, (D) and J exons occurring because of these processes result in large and unique immunoglobulin repertoires within individuals of an animal species although each individual may have have identical germline sequences.

The immunoglobulin-like binding polypeptides of the invention mimic immunoglobulins in both sequence diversity and immunological repertoire. Immunoglobulin-like binding polypeptides of the invention reconstruct the results of germline and junctional diversity in vivo recombination utilizing efficient synthetic or recombinant methods which produce random combinations of exon encoded V, (D) and J region family member sequences and which randomly vary the length and sequence at junctional regions. The immunoglobulin-like binding polypeptides of the invention also can incorporate arbitrary sequences at, for example, the V_{H}D or the DJ junctions to create diversity additional to that encoded in natural gene repertoires.

Immunoglobulin-like binding polypeptides of the invention include, for example, V_{H}-like, V_{L}-like and F_{V}-like binding polypeptides as well as functional fragments of these binding polypeptides. As described further below, F_{V}-like binding polypeptides are composed of heavy and light chain-like subunits through self-assembly similar to immunoglobulin F_{V} fragments. Association of heavy and light chain-like polypeptides into a dimeric or other multimeric tertiary polypeptide structure is maintained by non-covalent or covalent interactions similar to F_{V} fragments. Accordingly, the V_{H}-like and V_{L}-like subunit polypeptides forming a F_{V}-like binding polypeptides of the invention can include or not include interchain disulfide bonds depending on the length of the V_{H}-like or V_{L}-like subunit polypeptide. Similarly, all methods, molecular structures and other moieties employed with, or used in connection with the design or production of various modified antibody-like molecules known in the art can similarly be employed for the design or production of the immunoglobulin-like binding polypeptides of the invention.

Isolated populations of diverse V_{H}-like binding polypeptides are composed of random combinations of germline exon encoding sequences corresponding to V_{H}, D and J regions of a V_{H} polypeptide. Similarly, isolated populations of diverse V_{L}-like binding polypeptides are composed of random combinations of germline exon encoding sequences corresponding to V_{L} and J regions of a V_{L} polypeptide. Such combinations mimic in vivo germline diversity and can be designed to result in either exact joining of exon encoded sequences or imprecise joining to generate additional junctional diversity. Further junctional diversity can be generated by inclusion of known or arbitrary sequences at the junctional regions.

As described further below, the combinations of V, (D) and J region exon encoding sequences can be performed by enzymatic or chemical synthesis and expression of the encoding nucleic acids. Alternatively, the combinations can be generated by synthesis of the amino acid sequence. Synthesis and expression enables the efficient production of both large and small populations of immunoglobulin-like binding polypeptides. However, in some instances, it can be beneficial to directly synthesize one or a few immunoglobulin-like binding polypeptides depending on the intended use or the desired result to be achieved. Given the teachings and guidance provided herein, those skilled in the art will be able to select a desired method for the production of a diverse population of immunoglobulin-like binding polypeptides. The invention will be described herein with reference to the production diverse populations by construction of encoding nucleic acid sequences and expression into polypeptides. However, it is understood that the descriptions with reference to immunoglobulin-like encoding nucleic acids or construction thereof are similarly applicable to their corresponding immunoglobulin-like binding polypeptides by virtue of their relationship through the genetic code.

A diverse population of V_{H}-like binding polypeptides consists of unascertained combinations of exons encoding the complete V_{H}-like polypeptide. Similarly, a diverse population of V_{L}-like binding polypeptides consists of unascertained combinations of exons encoding the complete V_{L}-like polypeptide. Nucleic acids encoding some or all possible combinations of V_{H} or V_{L}, (D) and J_{H} or J_{L} region exon encoding sequences can be designed and synthesized as contiguous nucleic acid sequences. Alternatively, nucleic acids corresponding to the exon encoding regions can be synthesized and then assembled by, for example, ligation, annealing and ligation, or chemical coupling. The diversity of the resulting population will depend on the number of encoding exons to include as assembly members.

A diverse population of V_{H}-like binding polypeptides are designed by selecting sets of V_{H}, D and J exon family members to combine into contiguous sequences having the V_{H}-D-J structure of an V_{H} immunoglobulin region. For design of a single V_{H}-like binding polypeptide, the set will include one member from each exon family. For example, an encoding nucleic acid for a V_{H}-like binding polypeptide can include a combination corresponding to V_{H1}-D₁-J₁ exons. Design of a diverse population of V_{H}-like binding polypeptides can include small or large sets of exon family members. A small set can consist, for example, of two or more exon members from one family and a single family member from each of the other two families of encoding exon region sequences. For example, two V_{H} exon sequences can be joined with a D and J region exon sequence to result in a population encoding V_{H}-like binding polypeptides having the structures V_{H1}-D₁-J₁ and V_{H2}-D₁-J₁ exons. Alternatively, two D region encoding exons can be joined with a V_{H} and a J region exon, or two J region exons can be joined with a V_{H} and a D region exon, to yield the populations V_{H1}-D₁-J₁ and V_{H1}-D₂-J₁, and V_{H1}-D₁-J₁ and V_{H1}-D₁-J₂, respectively.

Diversity of a diverse population of V_{H}-like polypeptides can be increased by increasing the number of exon family members selected for an assembly set. Diversity is proportional to the number of possible combinations between V_{H}, D and J region encoding exons. Accordingly, as the number of family members increases so does the number of possible combinations between V_{H}, D and J regions that can be designed and generated. Larger assembly sets can consist, for example, of two or more members from each exon family or more than two members from one family and one or more members from the other two families. For example, combinations arising from the former example will yield populations containing the possible structures V_{H1,2,n}-D_{1,2,n}-J_{1,2,n} or at least 3² different possibilities which corresponds to at least six different species. Combinations arising from the latter example will yield populations containing the possible structures V_{H1,2,3,n}-D_{1,n}-J_{1,n} or at least six different species.

The subscripted numbers used in the above nomenclature represent the exon family members from 1 to n that can be found at the identified position. Briefly, with reference to the first example, V_{H1} and V_{H2} exons can be found within the population at the V_{H} exon encoded position, and each can be paired in combination with either D₁ or D₂ and either J₁ or J₂, yielding the at least six different possible combinations described above. Where n is greater than 2 exon family members the diversity of the population increases proportional to the number of additional members.

Accordingly, diverse assembly sets can consist, for example, of many different members from each family. Similarly, highly diverse assembly sets can consist, for example, of most or all exon members from each family. For example, the possible number of combinations arising from 51 different V_{H} exons, 25 different D region exons and 6 different J region exons is larger than 7,500 unique species. Other exemplary assembly sets and diversity of the resultant populations include, for example, small, medium and large populations with diversity between about 2-2000, 2001-15,000 and 15,001-50,000, respectively, or more than 50,000 different species. Using the compositions and methods of the invention diversities of 10³, 10⁴, 10⁵, 10⁶, 10⁷, 10⁸ or more can be readily generated.

A specific example of a set of V_{H}, D and J region exons for a small population diversity includes, for example, about 1 V_{H}, 2 D and 6 J region exons, which yields a diversity of about 12 or more different species. A specific example of a set of V_{H}, D and J region exons for a medium population diversity includes, for example, about 10 V_{H}, 5 D and 6 J region exons, which yields a diversity of about 3,000 or more different species. A specific example of a set of V_{H}, D and J region exons for a large population diversity includes, for example, about 200 V_{H}, 25 D and 6 J region exons, which yields a diversity of about 30,000 or more different species. Particularly, useful population diversities include, for example, a set of about 150 V_{H}, 1 D and 1 J region exons, which yields a diversity of about 150 different species, or a set of about 30 V_{H}, 5 D and 6 J region exons, which yields a diversity of about 9,000 different species. Given the teachings and guidance provided herein those skilled in the art will understand that all various combinations and permutations of V_{H}, D and J region exons can be combined to generate all diversity size ranges between, above and below the exemplary populations sizes set forth above.

Given the teachings and guidance provided herein, population diversity other than those exemplified above also can be generated by employing the requisite number of V_{H}, D and J exon members in an assembly set. Accordingly, population diversity can range from two V_{H}-like binding polypeptides to 10¹⁰ or greater. Similarly, population diversity can range from two V_{L}-like binding polypeptides to 10¹⁰ or greater. Similarly, all integer values in between these diversity sizes also can be generated by adjusting the number of component exons contained in the starting assembly set.

Combinations of exon encoding sequences are designed or produced to randomly join the component exons family members of the assembly set. The design can be manual or automated to yield some or all different and unascertained combinations or permutations of the component members of the assembly set. Production of a diverse population of unascertained combinations can be preformed by first generating the component members and then random assembly of the assembly members without predetermined design of the resultant species. In the former instance, molecules are designed and then synthesized. In the latter instance, molecules are first synthesized and then randomly joined. Either approach yields the same or similar population of unascertained combinations of V_{H}, D and J exon encoding sequences.

Similarly, a diverse population of V_{L}-like binding polypeptides are designed by selecting sets of V_{L} and J exon family members to combine into contiguous sequences having the V_{L}-J structure of an V_{L} immunoglobulin region. As with the design of a V_{H}-like binding polypeptide, the design of a V_{L}-like binding polypeptide will include in the assembly set members from each exon family. For example, an encoding nucleic acid for a single V_{L}-like binding polypeptide can include a combination corresponding to V_{L1}-J₁ exons. Design of a diverse population of V_{L}-like binding polypeptides can include small or large sets of exon family members as well as all V_{L} and J exon family members. V_{L} exon family members can include V_{κ}, V_{λ} or both. Similarly, J_{L} exon family members can include J_{κ}, J_{λ} or both. Accordingly, following the teachings and guidance provided above with reference to V_{H}-like binding polypeptide populations, different V_{L} and J family members can be utilized in an assembly set to produce populations having the structures V_{L1,2,n}-J_{1,2,n} where n represents the number of exon family members included in an assembly set and J region exons can be either J_{κ}, J_{λ} or both.

As with the V_{H}-like binding polypeptides described above, diversity of a resulting population of V_{L}-like polypeptides can be increased by increasing the number of exon family members selected for an assembly set because it will be proportional to the number of possible combinations between V_{L} and J region encoding exons. Small populations can include, for example, from 1-2 members of each exon family where there is at least a total of 3 exon family members from all families. Larger populations can include, for example, two or more members from each family whereas large populations will contain a plurality of members from each family and diverse as well as highly diverse populations can include, for example, many or all V_{L}, J_{κ} and J_{λ} exon encoding sequences. For example, the possible number of combinations arising from 70 different V_{L} exons 9 different J_{L} region exons is about 540. Other exemplary assembly sets and diversity of such resultant V_{L}-like binding polypeptide populations include, for example, small, medium and large populations with diversity between about 2-25, 26-200 and 201-800, respectively, or more than 50,000 different species. Using the compositions and methods of the invention diversities of V_{L}-like binding polypeptides of about 10², 10³, 10⁴, 10⁵, 10⁶, 10⁷, 10⁸ or more can be readily generated.

A specific example of a set of V_{L} and J region exons for a small population diversity includes, for example, about 1 V_{L} and 9 J region exons, which yields a diversity of about 9 or more different species. A specific example of a set of V_{L} and J region exons for a medium population diversity includes, for example, about 10 V_{L} and 5 J region exons, which yields a diversity of about 50 or more different species. A specific example of a set of V_{L} and J region exons for a large population diversity includes, for example, about 70 V_{L} and 9 J region exons, which yields a diversity of about 630 or more different species. Particularly, useful population diversities include, for example, a set of about 10 V_{L} and 1 J region exons, which yields a diversity of about 10 different species, or a set of about 60 V_{L} and 5 J region exons, which yields a diversity of about 300 different species. Given the teachings and guidance provided herein those skilled in the art will understand that all various combinations and permutations of V_{L} and J region exons can be combined to generate all diversity size ranges between, above and below the exemplary populations sizes set forth above.

Sequences that can be used to design or generate the V_{H}-like or V_{L}-like binding polypeptides of the invention include any animal immunoglobulin V_{H}, V_{L}, (D), J_{H}, or J_{L} exon encoding nucleic acid or corresponding amino acid sequence. Exemplary animal species include, for example, human, primate, murine, rat, other rodents, goat and porcine. Nucleic acid or amino acid sequences derived from human sources are beneficial because they can be directly employed as human therapeutics with minimal adverse effects resulting from host immune responses. The nucleotide sequence for the genomes of many of these species have been sequenced. Additionally, the nucleotide sequences and amino acid sequences of a large number of immunoglobulins has been the focus of investigations independent of genome sequencing projects. Therefore, the sequence and structure of immunoglobulins are well known to those skilled in the art. Any or all of such sequences can be used as members in an assembly set for the design or production of V_{H}-like or V_{L}-like binding polypeptides of the invention.

Briefly, nucleotide sequences and amino acid sequences for the about 51 human V_{H} exon sequences can be found in public sequence data bases. Moreover, these sequences also can be found described in Kabat et al., supra. The human nucleotide sequences for the about 51 different functional V_{H} exons are shown in Table 1 (SEQ ID NOS:1-51). Similarly, the nucleotide sequences and amino acid sequences for the about 70 V_{L} exon sequences as well as for the about 25 D, 6 J_{H} and 9 J_{L} exon sequences also can be found, for example, in public sequence databases or in Kabat et al., supra. The human nucleotide sequences for the about 25 different functional D region exons are shown in Table 2 (SEQ ID NOS:52-76). Table 3 shows the human nucleotide sequences for the about 6 different functional J_{H} region exons (SEQ ID NOS:77-82). Table 4 shows the human nucleotide sequences for the about 70 different functional V_{L} exons (SEQ ID NOS:83-152). These V_{L} exon sequences can be further divided into about 40 V_{κ} (SEQ ID NOS:83-122) and about 30 V_{H} (SEQ ID NOS:123-152) exon sequences. The human nucleotide sequences for the about 9 different functional J_{L} exons are shown in Table 5 (SEQ ID NOS:153-161). These J_{L} exon sequences can be further divided into about 5 different J_{κ} (SEQ ID NOS:153-157) and about 4 different J_{λ} exons (SEQ ID NOS:158-161).

The above sequences as well as those derived from species other than human can be used to design or generate unascertained combinations of V, (D) and J region exon sequences for the production of V_{H}-like or V_{L}-like binding polypeptides. Moreover, arbitrary sequences in place of, for example, any or some of the CDR1 or CDR2 region sequences of V_{H} or V_{L} exon encoding nucleic acids or in place of D or J exon encoding nucleic acid fragments can similarly be employed. The arbitrary sequences can be, for example, modeled after authentic CDR1, CDR2, D or J exon encoded polypeptides or they can partially or completely arbitrary. For example, some or all nucleotides or amino acid encoding positions can be randomized or unbiased. Alternatively, some or all nucleotides or amino acid encoding positions can be predetermined or biased toward predetermined residues. Additionally, arbitrary sequences can be used, for example, alone or in combination with known CDR1, CDR2, (D) or J exon encoded sequences to generate, for example, entirely different binding repertoires or to increase diversity of an available repertoire. Replacement or addition of binding domains within an immunoglobulin-like molecule of the invention with arbitrary sequences is are useful additional sources of binding domain region sequences because they replace one variable sequence with another variable sequence. Given the teachings and guidance provided herein, those skilled in the art will know or can determine when it can be beneficial to substitute any of CDR1, CDR2, (D) or J binding domains with partially or completely arbitrary nucleotide sequences or to utilize partially or completely arbitrary nucleotide sequences in addition to available V_{H}, V_{L}, (D) and J exon family members in an assembly set.

Junctional diversity can be introduced into V_{H}-like or V_{L}-like binding polypeptides by, for example, varying the length or sequence composition at any or all of the V, (D) or J junctional regions. Two junctional regions occur in V_{H}-like binding polypeptides because there are three exon regions that combine to form the complete V_{H}-like binding polypeptide. One junction occurs at the boundary where V_{H} and D exons are joined producing a V_{H}D junction. The second junction occurs at the boundary where D and J exons are joined producing a DJ junction. The intact V_{H}-like binding polypeptide will therefore contain the structure V_{H}DJ where junctional diversity can be generated at either or both of the V_{H}D or DJ junctions. By comparison, one junctional region occurs in a complete V_{L}-like binding polypeptides. This junctional region is where the V_{L} and J exons are joined to produce a V_{L}J junction.

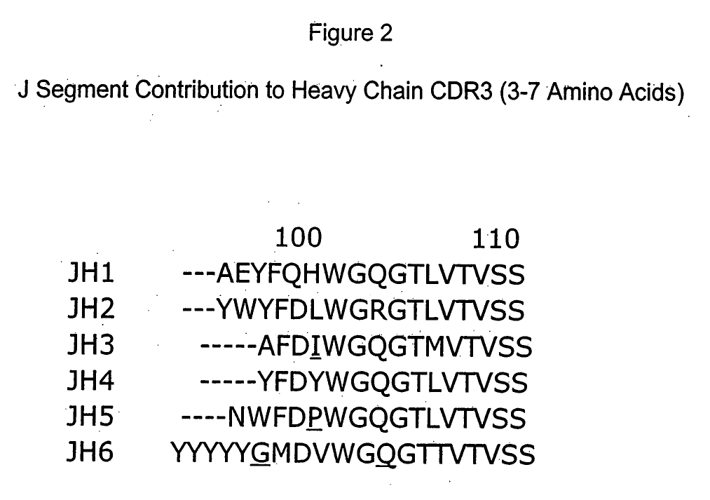

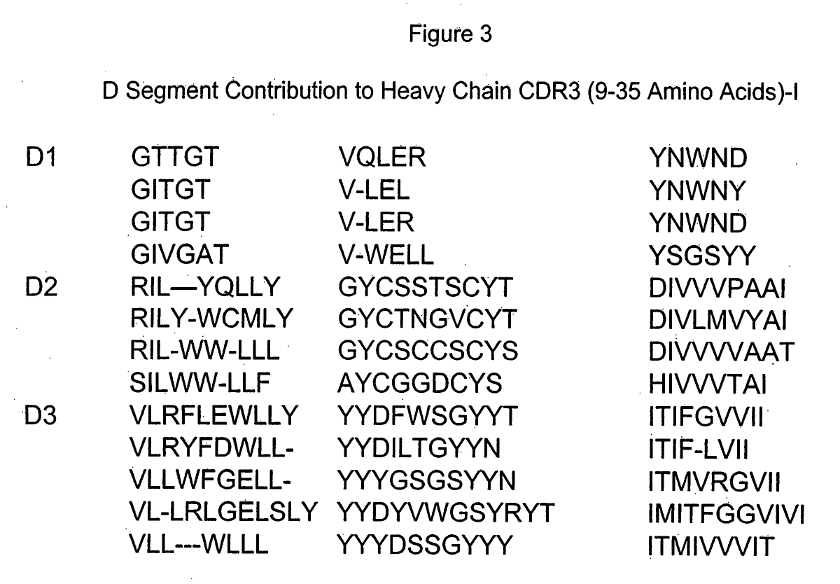

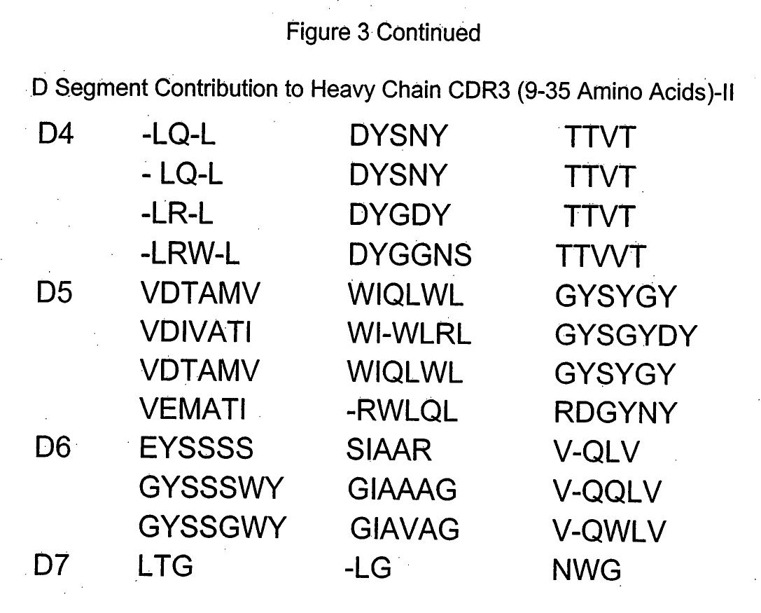

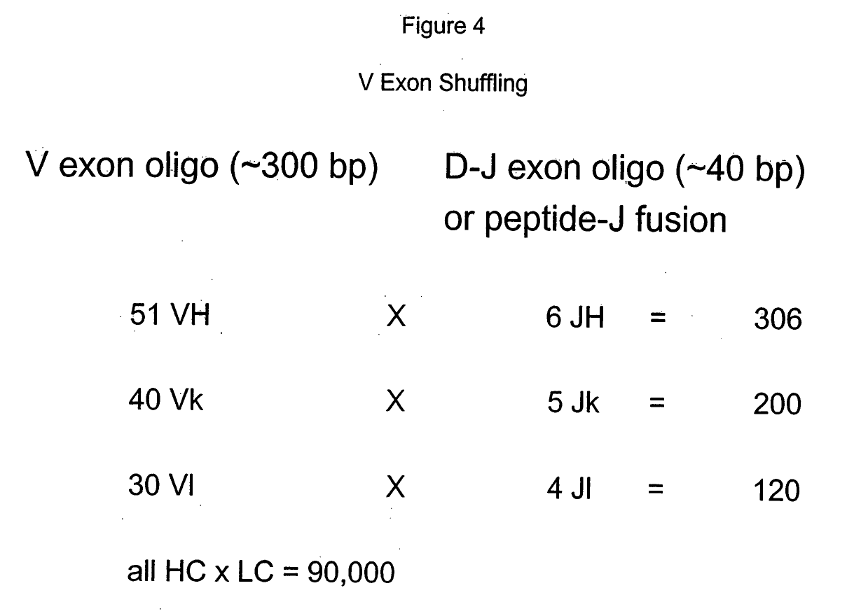

As described previously, D and J_{H} exons combine together and with a V_{H} exon to encode a portion of CDR3 region sequences of a V_{H}-like binding polypeptide. Similarly, a J_{L} exon combines with a V_{L} exon to encode a portion of V_{L}-like CDR3 region sequences. The contribution of (D) and J exons to CDR3 regions is shown in FIGS. 2 and 3. Briefly, J_{H} exons consist of about 9-21 nucleotides coding for about 3-7 amino acid residues (see also Table 3). Upon imprecise recombination J_{H} sequences provide represent between about 3-7 amino acids of a V_{H} CDR3 (FIG. 2). D region exons consist of about 27-105 nucleotides encoding a possible 9-35 amino acid residues (see also Table 2). Upon imprecise recombination of a D exon with both V_{H} and J_{H} exons the contribution of D exon sequences to V_{H} CDR3 is between about 9-35 amino acids (FIG. 3). Similar size contributions exist for J_{κ} and J_{λ} and are shown in, for example, Table 5. The V_{H}-like and V_{L}-like binding polypeptides of the invention incorporate similar size and sequence diversity in V(D)J junctional regions to create and augment the repertoire of immunoglobulin-like binding molecules of the invention.

Populations of V_{H}-like or V_{L}-like binding polypeptides can incorporate all possible ranges of (D) and J exon region sequence contribution found in naturally occurring immunoglobulin molecules. Populations can be created by arbitrarily including different size (D) or J region exons for family members constituting an assembly set. Alternatively, population diversity can be maximized by systematically including all possible sizes of (D) and/or J region exon sequences for some or all family members in an assembly set. For example, an assembly set including a D₁ family member exon which contributes between about 9-20 amino acids to V_{H} CDR3 can include size species of D₁ that fall within this range. The size species can be selected at random or they can represent different categorical sizes such as small, medium or large. In contrast, for example, the size species for inclusion within a D₁ containing assembly set also can include all possible sizes ranging from about 9-35 or more of the D₁ exon portion contributing to V_{H} CDR3. The size species for other D exon family members as well as for J_{H}, J_{κ} or J_{λ} or any combination of these exon regions or their family members similarly can be varied arbitrarily or systematically to include some or all possible size range variations mimicking junctional diversity occurring in vivo.

In addition to, or in lieu of, varying the size of (D) or J region exons, the sequence of these exons used in an assembly set also can be altered or substantially substituted. For example, where a (D) or J region exon contributes a certain range of amino acids to a V region CDR3, nucleic acids having a partially or completely arbitrary sequence but encompassing that amino acid range can be included in an assembly set. Incorporation of such random sequences into the (D) or J regions or both regions will yield sequence species within the population not previously included within an immunoglobulin repertoire. Given the teachings and guidance provided herein, those skilled in the art will know or can determine when it is beneficial to include arbitrary sequences in addition to, or in substitution of, available (D) or J region exon sequences.

Design of diverse populations of can be with reference to exon family member nucleotide sequence, amino acid sequence or both. As described previously and further below, production of diverse populations of V_{H}-like or V_{L}-like binding polypeptides can be by chemical or enzymatic synthesis of the encoding nucleic acids or chemical synthesis of the polypeptides. An alternative to design and synthesis is to generate the component exon members of an assembly set and then promote semi-random exon assembly by hybridization, enzymatic or chemical ligation or both. Semi-random assembly refers to an ordered 5′ to 3′, or amino to carboxyl terminal, assembly of V, (D) and J exon sequences but random incorporation of the exon family members of an assembly being incorporated into its respective exon position. Accordingly, once the encoding nucleic acids are designed and created or generated de novo they can be translated into the V_{H}-like or V_{L}-like polypeptides of the invention. Diverse populations of encoding nucleic acids can be translated in vitro or in vivo in host cells, tissue cultures or organisms. Methods of generating host cell libraries for propagation or expression of such encoding nucleic acid populations are well known in the art and are described further below.

Therefore, the invention provides isolated populations of V_{H}-like or V_{L}-like binding polypeptides (D) or J exon encoded polypeptides are derived from a human immunoglobulin amino acid sequence. The V_{H}-like populations can contain any unascertained combination of the about 51 different human V_{H} region encoded exons, the about 6 different human J_{H} region encoded exons and the about 25 different human D region encoded exons. The V_{L}-like populations can contain any unascertained combination of the about 70 different human V_{L} region encoded exons and the about 9 different J_{L} region encoded exons. Populations of V_{H}-like binding polypeptides can include diversities of at least about 10⁶. Diversities of at least about 10⁴, 10⁵ or greater also are useful. Populations of V_{L}-like binding polypeptides can include diversities of at least about 10⁶. Diversities of V_{L}-like binding polypeptides of at least about 10³ also are useful.

The invention further provides an isolated diverse population of F_{V}-like binding polypeptides, each binding polypeptide within the population comprising an unascertained combination of a V_{H}-like binding polypeptide and a V_{L}-like binding polypeptide, each of the V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of the V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein the V_{H}-like and the V_{L}-like binding polypeptides associate to form an immunoglobulin F_{V}-like binding polypeptide.

F_{V}-like binding polypeptides consist of at least one V_{H}-like and one V_{L}-like binding polypeptide. The V_{H}-like and V_{L}-like subunits can self-assemble into a dimeric or multimeric complex similar to F_{V} immunoglobulin polypeptides. Self-assembly of V_{H}-like and V_{L}-like into F_{V}-like binding polypeptides can occur simultaneous with expression or translation into their respective polypeptides from encoding nucleic acids. One efficient method for producing the F_{V}-like binding polypeptides of the invention is to coexpress in vitro or in vivo individual diverse populations of V_{H}-like and V_{L}-like binding polypeptides. The populations can be expressed, for example, from separate vector populations or the V_{H}-like and V_{L}-like can be combined into a single vector population where each member of the population harbors an unascertained V_{H}-like and V_{L}-like member of the individual populations. Alternatively, the F_{V}-like binding polypeptides of the invention can be produced by mixing a population of V_{H}-like binding polypeptides with a population of V_{L}-like binding polypeptides. The V_{H}-like and V_{L}-like binding polypeptides within the mixed population will subsequently assemble into, for example, dimeric F_{V}-like binding polypeptides mimicking F_{V} immunoglobulin fragments. Non-covalent and covalent interactions such as disulfide bonds promote assembly or stability of the F_{V}-like binding polypeptides similar to F_{V} immunoglobulin fragments because of their similar primary, secondary and tertiary stability.

Isolated populations of functional fragments for any of the previously described immunoglobulin-like binding polypeptides are also provided. Functional fragments can be any truncated immunoglobulin-like polypeptide of the invention so long as the fragment exhibits discriminatory binding activity toward the target ligand bound by the intact immunoglobulin-like binding polypeptide. The structure of immunoglobulins and functional fragments of immunoglobulins are well known to those skilled in the art. Any of such immunoglobulin functional fragments similarly can be generated from the immunoglobulin-like binding polypeptides of the invention.

For example, it has been shown that fragments corresponding to single CDR region of a parent immunoglobulin can exhibit discriminatory binding activity toward the target ligand bound by the parent immunoglobulin. Similarly, V_{H} and V_{L} polypeptides are known to exhibit discriminatory binding activity in the absence of pairing to form a V_{H}V_{L} dimeric structure. Fragments of V_{H}-like and V_{L}-like binding polypeptides corresponding to F_{d} chains are similarly included as functional fragments of the invention so long as they exhibit discriminatory binding to a target ligand. Similarly, an F_{d}-like dimeric molecule generated from F_{d} fragments of V_{H}-like and V_{L}-like polypeptides also are included as functional fragments of the invention. Those skilled in the art will know or can determine what fragment or fragments of the V_{H}-like, V_{L}-like or F_{V}-like binding polypeptides of the invention will maintain discriminatory binding toward a target ligand as does the intact immunoglobulin-like binding polypeptide from which it derives. All of such functional fragments exhibiting discriminatory binding are included as immunoglobulin-like binding polypeptides of the invention.

Any of the immunoglobulin-like binding polypeptides of the invention can be isolated for use in a variety of research, diagnostic or therapeutic procedures. Methods for isolation are well known to those skilled in the art. Moreover, and as described further below, the populations of immunoglobulin-like binding polypeptides can be screened for polypeptide species having a desired: binding activity. Identified binders can be isolated as a procedural step in the screening or the encoding nucleic acids can be, for example, identified and expressed in a host cell. These and other methods well known in the art can be used to generate a substantially pure immunoglobulin-like binding polypeptides of the invention.

Therefore, the invention provides a substantially pure V_{H}-like binding polypeptide consisting of an unascertained combination of an immunoglobulin V_{H} region exon encoded polypeptide, a J_{H} region exon encoded polypeptide and a D region exon encoded polypeptide, wherein the V_{H}, D and J_{H} region exon encoded polypeptides are joined in a single polypeptide forming an immunoglobulin V_{H}-like binding polypeptide. Further provided is a substantially pure V_{L}-like binding polypeptide consisting of an unascertained combination of an immunoglobulin V_{L} region exon encoded polypeptide and a J_{L} region exon encoded polypeptide, wherein the V_{L} region exon encoded polypeptide and the J_{L} region polypeptide are joined in a single polypeptide forming an immunoglobulin V_{L}-like binding polypeptide, or a functional fragment thereof. Substantially pure F_{V}-like binding polypeptide consisting of a V_{H}-like binding polypeptide and a V_{L}-like binding polypeptide, wherein the V_{H}-like and the V_{L}-like binding polypeptides associate to form an immunoglobulin F_{V}-like binding polypeptide also is provided by the invention. Substantially pure functional fragments of V_{H}-like, V_{L}-like and F_{V}-like binding polypeptides are further provided by the invention.

A immunoglobulin-like binding polypeptide can contain additional polypeptide sequences, structures and moieties so long as there is at least one immunoglobulin-like binding polypeptide or functional fragment thereof contained within the complex or structure. For example, a immunoglobulin-like binding polypeptide can consist of a immunoglobulin-like binding polypeptide and one or more domains or polypeptides that impart another function onto the immunoglobulin-like binding polypeptide. Accordingly, the immunoglobulin-like binding polypeptides of the invention can exhibit multiple functions, one of which, is binding to a target ligand. Functions other than binding activity of the immunoglobulin-like binding polypeptide similarly can include, for example, an activity, a structural feature or any other property inherent in an amino acid sequence. Additionally, functions associated with other macromolecules, organic compounds or inorganic compounds also can be imparted onto a immunoglobulin-like binding polypeptide of the invention by joinder of such molecules to the immunoglobulin-like binding polypeptide.

Multiple functions also can be conferred onto a immunoglobulin-like binding polypeptide through the construction of multimeric immunoglobulin-like binding polypeptides. As with the attachment or joinder of functional domains, polypeptides, macromolecules, or compounds to a immunoglobulin-like binding polypeptide to confer secondary characteristics onto a immunoglobulin-like binding polypeptide, two or more immunoglobulin-like binding polypeptides can be attached or joined as components of a multimeric immunoglobulin-like binding polypeptide. For example, two or more immunoglobulin-like binding polypeptides can be joined in linear or branched form to produce a dimer, trimer or other multimer of immunoglobulin-like binding polypeptides. Monomers of the multimers can exhibit the same or different binding activities toward one or more target ligands.

As described above and below, approaches and methods for constructing immunoglobulin-like binding polypeptides with or without the inclusion of additional polypeptide sequences, structures and moieties are well known in the art. For example, approaches can include insertion, substitution or directed changes of amino acid sequences. Approaches for inclusion of secondary functional characteristics can include, for example, the or attachment of functional domains, polypeptides or other moieties. Methods to implement such approaches can include, for example, recombinant construction and in vitro or in vivo synthesis, chemical synthesis, conjugation, linkers as well as the use of domains corresponding to affinity binding partners. Various other approaches and methods well known in the art can similarly be utilized to design and construct a immunoglobulin-like binding polypeptide of the invention.

Methods for constructing an immunoglobulin-like encoding nucleic acid and populations of such encoding nucleic acids are well known in the art. For example, encoding nucleic acids can be produced by any method of nucleic acid synthesis known to those skilled in the art. Such methods include, for example, chemical synthesis, recombinant synthesis, enzymatic polymerization and combinations thereof. These and other synthesis methods are well known to those skilled in the art.

For example, methods for synthesizing polynucleotides can be found described in, for example, Oligonucleotide Synthesis: A Practical Approach, Gate, ed., IRL Press, Oxford (1984); Weiler et al., Anal. Biochem. 243:218 (1996); Maskos et al., Nucleic Acids Res. 20:1679 (1992); Atkinson et al., Solid-Phase Synthesis of Oligodeoxyribonucleotides by the Phosphitetriester Method, in Oligonucleotide Synthesis 35 (M. J. Gait ed., 1984); Blackburn and Gait (eds.), Nucleic Acids in Chemistry and Biology, Second Edition, New York: Oxford University Press (1996), and in Ansubel et al., Current Protocols in Molecular Biology, John Wiley and Sons, Baltimore, Md. (1999).

Recombinant and enzymatic synthesis, including polymerase chain reaction and other amplification methodologies can be found described in, for example, Sambrook et al., Molecular Cloning: A Laboratory Manual, Third Ed., Cold Spring Harbor Laboratory, New York (2001) and in Ansubel et al., (1999), supra.

Solid-phase synthesis methods for generating numerous different polynucleotides and other polymer sequences can be found described in, for example, Pirrung et al., U.S. Pat. No. 5,143,854 (see also PCT Application No. WO 90/15070), Fodor et al., PCT Application No. WO 92/10092; Fodor et al., Science 251:767-777 (1991); Winkler et al., U.S. Pat. No. 6,136,269; Southern et al. PCT Application No. WO 89/10977, and Blanchard PCT Application No. WO 98/41531. Such methods include synthesis and printing of arrays using micropins, photolithography and ink jet synthesis of polynucleotide arrays. Methods for efficient synthesis of nucleic acid polymers by sequential annealing of polynucleotides can be found described in, for example, in U.S. Pat. No. 6,521,437, to Evans.

Chemical synthesis of polypeptides also is well known to those skilled in the art. Accordingly, a immunoglobulin-like binding polypeptide or populations of immunoglobulin-like binding polypeptides of the invention can be synthesized directly using any of a variety of methods well known in the art. Methods well known in the art for synthesizing peptides, polypeptides, peptidomimetics and proteins can be found described in, for example, U.S. Pat. Nos. 5,420,109; 5,849,690; 5,686,567; 5,990,273; PCT publication WO 01/00656; M. Bodanzsky, Principles of Peptide Synthesis (1st ed. & 2d rev. ed.), Springer-Verlag, New York, N.Y. (1984 & 1993) (see, for example, Chapter 7), and in Stewart and Young, Solid Phase Peptide Synthesis, (2d ed.), Pierce Chemical Co., Rockford, Ill. (1984).

Methods and chemistry for incorporating restrictive amino acid conformations, amino acid analogs, mimetics, bridges, and synthetic linkers also are well known in the art and can be used in the synthesis or modifications of populations or substantially pure immunoglobulin-like binding polypeptides of the invention. Specific examples of amino acid analogs and mimetics that can be useful in such approaches can be found described in, for example, Roberts and Vellaccio, The Peptides: Analysis, Synthesis, Biology, Eds. Gross and Meinhofer, Vol. 5, p. 341, Academic Press, Inc., New York, N.Y. (1983). Other examples include peralkylated amino acids, particularly permethylated amino acids, which can be found described in, for example, Combinatorial Chemistry, Eds. Wilson and Czarnik, Ch. 11, p. 235, John Wiley & Sons Inc., New York, N.Y. (1997). Yet other examples include amino acids whose amide portion (and, therefore, the amide backbone of the resulting peptide) has been replaced, for example, by a sugar ring, steroid, benzodiazepine or carbo cycle. Methods for the synthesis of alternatives for amide backbones can be found described in, for example, Burger's Medicinal Chemistry and Drug Discovery, Ed. Manfred E. Wolff, Ch. 15, pp. 619-620, John Wiley & Sons Inc., New York, N.Y. (1995).

The above exemplary synthesis methods and functionally equivalent structures are all well known to those skilled in the art and can be used in the production of isolated populations or substantially pure immunoglobulin-like encoding nucleic acids or polypeptides. Any of these methods can be used, for example, alone or in combination with other methods of synthesis well known in the art. Given the teachings and guidance provided herein, those skilled in the art will know or can determine which method of synthesis is appropriate for the generation of encoding nucleic acids or binding polypeptides populations of the invention.

Once the populations of immunoglobulin-like encoding nucleic acids have been constructed as described above, they can be expressed to generate a population of V_{H}-like, V_{L}-like or F_{V}-like binding polypeptides that can be screened for binding affinity to a target ligand. For example, encoding nucleic acids for immunoglobulin-like binding polypeptides can be synthesized or cloned into an appropriate vector for propagation, manipulation and expression. Vectors conferring such functions are well known in the art or can be constructed by those skilled in the art. Generally, expression vectors will contain expression elements sufficient for the transcription, translation, regulation, and if desired, sorting and secretion of the altered scaffold polypeptides. Vectors sufficient for propagation can omit the expression components. The vectors also can be for use in either procaryotic or eukaryotic host systems so long as the expression and regulatory elements are of compatible origin. Expression vectors can additionally include regulatory elements for inducible or cell type-specific expression. Those skilled in the art will know which host systems are compatible with a particular vector and which regulatory or functional elements are sufficient to achieve expression of the immunoglobulin-like binding polypeptides in soluble, secreted or cell surface forms.

Appropriate host cells include, for example, bacteria and corresponding bacteriophage expression systems, yeast, avian, insect and mammalian cells. Methods for recombinant expression of isolated populations of V_{H}-like, V_{L}-like or F_{V}-like binding polypeptides in various host systems are well known in the art and are described, for example, in Sambrook et al., supra, and in Ansubel et al., supra. Methods for screening or purification of isolated populations of immunoglobulin-like binding polypeptides or for individual binders within such populations similarly are well known to those skilled in the art. The choice of a particular vector and host system for expression or for screening of immunoglobulin-like binding polypeptide of the invention will be known by those skilled in the art and will depend on the preference of the user.

The expressed populations of immunoglobulin-like binding polypeptides can be screened for the identification of one or more binding polypeptides exhibiting a selective binding affinity to a target ligand. Isolated populations of V_{H}-like or V_{L}-like binding polypeptides can be, for example, expressed alone and screened for binding affinity to a target ligand. Alternatively, isolated populations of V_{H}-like and V_{L}-like binding polypeptides can be coexpressed so that they self-assemble into F_{V}-like binding polypeptides. The multimeric F_{V}-like binding polypeptides containing unascertained combinations of V_{H}-like and V_{L}-like binding polypeptides can be screened for species exhibiting specific binding affinity to one or more target ligands. A specific example of the coexpression of V_{H}-like and V_{L}-like binding polypeptides into a diverse population of F_{V}-like binding polypeptides is described further below in the examples.

Screening of any of the isolated populations of V_{H}-like, V_{L}-like or F_{V}-like binding polypeptides for specific binding to a target ligand can be accomplished using various methods well known in the art for determining binding affinity of a polypeptide or compound. Additionally, methods based on determining the relative affinity of binding molecules to their partner by comparing the amount of binding between, for example, two or more immunoglobulin-like binding polypeptides or between one or more immunoglobulin-like binding polypeptides and a reference immunoglobulin can similarly be used for the identification of a predetermined binding species.

All of such methods can be performed, for example, in solution or in solid phase. Moreover, various formats of binding assays are well known in the art and include, for example, immobilization to filters such as nylon or nitrocellulose; two-dimensional arrays, enzyme linked immunosorbant assay (ELISA), radioimmune assay (RIA), panning and plasmon resonance. Such methods can be found described in, for example, Sambrook et al., supra, and Ansubel et al., supra. Methods for measuring the affinity, including association and disassociation rates using surface plasmon resonance are well known in the art and can be found described in, for example, Jonsson and Malmquist, Advances in Biosnsors, 2:291-336 (1992) and Wu et al. Proc. Natl. Acad. Sci. USA, 95:6037-6042 (1998). Moreover, one apparatus well known in the art for measuring binding interactions is a BIAcore 2000 instrument which is commercially available through Pharmacia Biosensor, (Uppsala, Sweden).

Using any of the above described screening methods, as well as others well known in the art, an V_{H}-like, V_{L}-like, F_{V}-like or functional fragments thereof can be identified by detecting the binding of at least one immunoglobulin-like binding polypeptide within the population to a target antigen. Additionally, the above methods can alternatively be modified by, for example, the addition of substrate and reactants to identify altered variable regions having a predetermined catalytic activity. Those skilled in the art will know, or can determine, binding conditions which are sufficient to identify selective interactions over non-specific binding.

Detection methods for identification of binding species within a population of immunoglobulin-like binding polypeptides of the invention can be direct or indirect and can include, for example, the measurement of light emission, radioisotopes, colorimetric dyes and fluorochromes. Direct detection includes methods that operate without intermediates or secondary measuring procedures to assess the amount of bound antigen or ligand. Such methods generally employ ligands that are themselves labeled by, for example, radioactive, light emitting or fluorescent moieties. In contrast, indirect detection includes methods that operate through an intermediate or secondary measuring procedure. These methods generally employ molecules that specifically react with the antigen or ligand and can themselves be directly labeled or detected by a secondary reagent. For example, an immunoglobulin-like binding polypeptide specific for a target ligand can be detected using a secondary antibody capable of interacting with the binding polypeptide specific for the ligand, again using the detection methods described above for direct detection. Indirect methods can additionally employ detection by enzymatic labels. Moreover, for the specific example of screening for a catalytic immunoglobulin-like binding polypeptide, the disappearance of a substrate or the appearance of a product can be used as an indirect measure of binding affinity or catalytic activity.

The methods of synthesis, expression, screening and detection described above are equally applicable to populations of V_{H}-like, V_{L}-like or F_{V}-like binding polypeptides. Similarly, the above described methods also are applicable to small, medium, large and very diverse size populations of immunoglobulin-like binding polypeptides of the invention. Those skilled in the art will known, or can determine using the teachings and guidance provided herein, which methods can be used to construct or facilitate the synthesis or identification of any of the various immunoglobulin-like binding polypeptides of the invention, or functional fragments thereof.

Therefore, the invention provides a method of identifying a F_{V}-like binding polypeptide having a predetermined binding activity. The method consists of: (a) contacting an isolated diverse population of F_{V}-like binding polypeptides with a test compound under conditions sufficient for binding, each of the F_{V}-like binding polypeptides comprising an unascertained combination of a V_{H}-like binding polypeptide and a V_{L}-like binding polypeptide, the V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of the V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein the V_{H}-like and the V_{L}-like binding polypeptides associate to form an immunoglobulin F_{V}-like binding polypeptide, and (b) measuring binding of one or more members in the population, wherein specific binding of a member in the population to the test compound identifies a F_{V}-like binding polypeptide having test compound specific binding activity.

Further provided is a method of producing an isolated diverse population of F_{V}-like binding polypeptides. The method consists of coexpressing a first population of nucleic acids encoding a diverse population of V_{H}-like binding polypeptides and a second population of nucleic acids encoding a diverse population of V_{L}-like binding polypeptides, each of the V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of the V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein the coexpressed first and second populations of binding polypeptides coassemble into unascertained V_{H}-like and V_{L}-like binding polypeptide combinations forming a diverse population of F_{V}-like binding polypeptides.

It is understood that modifications which do not substantially affect the activity of the various embodiments of this invention are also included within the definition of the invention provided herein. Accordingly, exemplary descriptions of specific embodiments are intended to illustrate but not limit the present invention.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. An isolated diverse population of V_{H}-like binding polypeptides, each binding polypeptide within said population comprising an unascertained combination of an immunoglobulin V_{H} region exon encoded polypeptide, a J_{H} region exon encoded polypeptide and a D region exon encoded polypeptide, wherein said V_{H}, D and J_{H} region exon encoded polypeptides are joined in a single polypeptide forming an immunoglobulin V_{H}-like binding polypeptide, or a functional fragment thereof.

2. The isolated population of V_{H}-like binding polypeptides of claim 1, wherein said V_{H} region, J_{H} region or D region exon encoded polypeptides are derived from a human immunoglobulin amino acid sequence.

3. The isolated population of V_{H}-like binding polypeptides of claim 1, wherein said immunoglobulin V_{H} region exon is selected from a family of about 51 different human V_{H} region encoded exons.

4. The isolated population of V_{H}-like binding polypeptides of claim 1, wherein said J_{H} region exon encoded polypeptide is selected from a family of about 6 different human J_{H} region encoded exons.

5. The isolated population of V_{H}-like binding polypeptides of claim 1, wherein said D region exon encoded polypeptide is selected from a family of about 25 different human D region encoded exons.

6. The isolated population of V_{H}-like binding polypeptides of claim 1, wherein said diverse population comprises a diversity of at least about 2-5, preferably at least about 100-499, more preferably 500 or more.

7. The isolated population of V_{H}-like binding polypeptides of claim 1, wherein said unascertained combination comprises any combination of a V_{H} region exon encoded polypeptide, a J_{H} region exon encoded polypeptide and a D region exon encoded polypeptide joined into a single polypeptide species.

8. An isolated diverse population of V_{L}-like binding polypeptides, each binding polypeptide within said population comprising an unascertained combination of an immunoglobulin V_{L} region exon encoded polypeptide and a J_{L} region exon encoded polypeptide, wherein said V_{L} region exon encoded polypeptide and said J region exon encoded polypeptide are joined in a single polypeptide forming an immunoglobulin V_{L}-like binding polypeptide, or a functional fragment thereof.

9. The isolated population of V_{L}-like binding polypeptides of claim 8, wherein said V_{L} region or J_{L} region exon encoded polypeptides are derived from a human immunoglobulin amino acid sequence.

10. The isolated population of V_{L}-like binding polypeptides of claim 8, wherein said immunoglobulin V_{L} region exon is selected from a family of about 70 different human V_{L} region encoded exons.

11. The isolated population of V_{L}-like binding polypeptides of claim 10, wherein said family of human V_{L} region encoded exons comprise about 40 different V_{κ} encoded exons.

12. The isolated population of V_{L}-like binding polypeptides of claim 10, wherein said family of human V_{L} region encoded exons comprise about 30 different V_{λ} encoded exons.

13. The isolated population of V_{L}-like binding polypeptides of claim 8, wherein said J_{L} region exon encoded polypeptide portion is selected from a family of about 9 different human J_{L} region encoded exons.

14. The isolated population of V_{L}-like binding polypeptides of claim 13, wherein said family of human V_{L} region encoded exons comprise about 5 different J_{κ} encoded exons.

15. The isolated population of V_{L}-like binding polypeptides of claim 13, wherein said family of human V_{L} region encoded exons comprise about 4 different J_{λ} encoded exons.

16. The isolated population of V_{L}-like binding polypeptides of claim 8, wherein said diverse population comprises a diversity of at least about 2-5, preferably at least about 100-499, more preferably 500 or more.

17. The isolated population of V_{L}-like binding polypeptides of claim 8, wherein said unascertained combination comprises any combination of a V_{L} region exon encoded polypeptide and a J_{L} region exon encoded polypeptide joined into a single polypeptide species.

18. An isolated diverse population of F_{V}-like binding polypeptides, each binding polypeptide within said population comprising an unascertained combination of a V_{H}-like binding polypeptide and a V_{L}-like binding polypeptide, each of said V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of said V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein said V_{H}-like and said V_{L}-like binding polypeptides associate to form an immunoglobulin F_{V}-like binding polypeptide.

19. The isolated population of F_{V}-like binding polypeptides of claim 18, wherein said V_{H}-like binding polypeptide comprises a V_{H} region, J_{H} region or D region exon encoded polypeptides derived from a human immunoglobulin amino acid sequence.

20. The isolated population of F_{V}-like binding polypeptides of claim 18, wherein said single V_{H}-like binding polypeptide comprises any unascertained combination of a V_{H} region exon encoded polypeptide, a J_{H} region exon encoded polypeptide and a D region exon encoded polypeptide.

21. The isolated population of F_{V}-like binding polypeptides of claim 18, wherein said V_{L}-like binding polypeptide comprises a V_{L} region or J_{L} region exon encoded polypeptide derived from a human immunoglobulin amino acid sequence.

22. The isolated population of F_{V}-like binding polypeptides of claim 18, wherein said single V_{L}-like binding polypeptide comprises any unascertained combination of a V_{L} region exon encoded polypeptide and a J_{L} region exon encoded polypeptide.

23. The isolated population of F_{V}-like binding polypeptides of claim 18, wherein said diverse population comprises a diversity of at least about 2-5, preferably at least about 100-499, more preferably 500 or more.

23. The isolated population of F_{V}-like binding polypeptides of claim 18, wherein said function fragments of said V_{H}-like or said V_{L}-like fusing binding polypeptides associate to form a immunoglobulin-like F_{d} fragment.

24. A substantially pure V_{H}-like binding polypeptide, comprising an unascertained combination of an immunoglobulin V_{H} region exon encoded polypeptide, a J_{H} region exon encoded polypeptide and a D region exon encoded polypeptide, wherein said V_{H}, D and J_{H} region exon encoded polypeptides are joined in a single polypeptide forming an immunoglobulin V_{H}-like binding polypeptide, or a functional fragment thereof.

25. The substantially pure V_{H}-like binding polypeptides of claim 24, wherein said V_{H} region, J_{H} region or D region exon encoded polypeptides are derived from a human immunoglobulin amino acid sequence.

26. The substantially pure V_{H}-like binding polypeptides of claim 24, wherein said immunoglobulin V_{H} region exon is selected from a family of about 51 different human V_{H} region encoded exons.

27. The substantially pure V_{H}-like binding polypeptides of claim 24, wherein said J_{H} region exon encoded polypeptide portion is selected from a family of about 6 different human J_{H} region encoded exons.

28. The substantially pure V_{H}-like binding polypeptides of claim 24, wherein said D region exon encoded polypeptide portion is selected from a family of about 25 different human D region encoded exons.

29. The substantially pure V_{H}-like binding polypeptides of claim 24, wherein said unascertained combination comprises any combination of a V_{H} region exon encoded polypeptide, a J_{H} region exon encoded polypeptide and a D region exon encoded polypeptide joined into a single polypeptide species.

30. A substantially pure V_{L}-like binding polypeptide, comprising an unascertained combination of an immunoglobulin V_{L} region exon encoded polypeptide and a J_{L} region exon encoded polypeptide, wherein said V_{L} region exon encoded polypeptide and said J_{L} region polypeptide are joined in a single polypeptide forming an immunoglobulin V_{L}-like binding polypeptide, or a functional fragment thereof.

31. The substantially pure V_{L}-like binding polypeptides of claim 30, wherein said V_{L} region or J_{L} region exon encoded polypeptides are derived from a human immunoglobulin amino acid sequence.

32. The substantially pure V_{L}-like binding polypeptides of claim 30, wherein said immunoglobulin V_{L} region exon is selected from a family of about 70 different human V_{L} region encoded exons.

33. The substantially pure V_{L}-like binding polypeptides of claim 32, wherein said family of human V_{L} region encoded exons comprise about 40 different V_{κ} encoded exons.

34. The substantially pure V_{L}-like binding polypeptides of claim 32, wherein said family of human V_{L} region encoded exons comprise about 30 different V_{λ} encoded exons.

35. The substantially pure V_{L}-like binding polypeptides of claim 30, wherein said J_{L} region exon encoded polypeptide portion is selected from a family of about 9 different human J_{L} region encoded exons.

36. The substantially pure V_{L}-like binding polypeptides of claim 35, wherein said family of human V_{L} region encoded exons comprise about 5 different J_{κ} encoded exons.

37. The substantially pure V_{L}-like binding polypeptides of claim 35, wherein said family of human V_{L} region encoded exons comprise about 4 different J_{λ} encoded exons.

38. The substantially pure V_{L}-like binding polypeptides of claim 30, wherein said unascertained combination comprises any combination of a V_{L} region exon encoded polypeptide and a J_{L} region exon encoded polypeptide joined into a single polypeptide species.

39. A substantially pure F_{V}-like binding polypeptide, comprising a V_{H}-like binding polypeptide and a V_{L}-like binding polypeptide, said V_{H}-like binding polypeptide comprising an unascertained combination of an immunoglobulin V_{H} region exon encoded polypeptide, a J_{H} region exon encoded polypeptide and a D region exon encoded polypeptide joined in a single polypeptide, or a functional fragment thereof, said V_{L}-like binding polypeptide comprising an unascertained combination of an immunoglobulin V_{L} region exon encoded polypeptide, and a J_{L} region exon encoded polypeptide joined in a single polypeptide, or a functional fragment thereof, wherein said V_{H}-like and said V_{L}-like binding polypeptides associate to form an immunoglobulin F_{V}-like binding polypeptide.

40. The substantially pure F_{V}-like binding polypeptides of claim 39, wherein said V_{H}-like binding polypeptide comprises a V_{H} region, J_{H} region or D region exon encoded polypeptides derived from a human immunoglobulin amino acid sequence.

41. The substantially pure F_{V}-like binding polypeptides of claim 39, wherein said single V_{H}-like binding polypeptide comprises any unascertained combination of a V_{H} region exon encoded polypeptide, a J_{H} region exon encoded polypeptide and a D region exon encoded polypeptide.

42. The substantially pure F_{V}-like binding polypeptides of claim 39, wherein said V_{L}-like binding polypeptide comprises a V_{L} region or J_{L} region exon encoded polypeptide derived from a human immunoglobulin amino acid sequence.

43. The substantially pure F_{V}-like binding polypeptides of claim 39, wherein said single V_{L}-like binding polypeptide comprises any unascertained combination of a V_{L} region exon encoded polypeptide and a J_{L} region exon encoded polypeptide.

44. The substantially pure F_{V}-like binding polypeptides of claim 39, wherein said functional fragments of said V_{H}-like or said V_{L}-like fusing binding polypeptides associate to form a immunoglobulin-like F_{d} fragment.

45. A method of identifying a F_{V}-like binding polypeptide having a predetermined binding activity, comprising:
(a) contacting an isolated diverse population of F_{V}-like binding polypeptides with a test compound under conditions sufficient for binding, each of said F_{V}-like binding polypeptides comprising an unascertained combination of a V_{H}-like binding polypeptide and a V_{L}-like binding polypeptide, said V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of said V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein said V_{H}-like and said V_{L}-like binding polypeptides associate to form an immunoglobulin F_{V}-like binding polypeptide, and
(b) measuring binding of one or more members in said population, wherein specific binding of a member in said population to said test compound identifies a F_{V}-like binding polypeptide having test compound specific binding activity.

46. The method of claim 45, wherein said test compound is selected from the group consisting of polypeptide, nucleic acid, lipid, carbohydrate and small organic molecule.

47. The method of claim 46, wherein said polypeptide is selected from the group consisting of IL2, IL12, IL6, fibrin, thrombin, growth hormone, prolactin, carcinoembryonic antigen.

48. The method of claim 46, wherein said nucleic acid is selected from the group consisting of nucleic acid binding polypeptides, transcription regulators and translation regulators.

49. The method of claim 46, wherein said lipid is selected from the group consisting of phosphoinositol, long chain saturated hydrocarbons, fatty acid aldehydes, cholesterol and a lipid anchored membrane polypeptide.

50. The method of claim 46, wherein said carbohydrate is selected from the group consisting of glucoseamine, heparin sulfate, galactosamine, glycogen, a cell surface proteogylcan and a cell surface glycoprotein.

51. The method of claim 46, wherein said small organic molecule is selected from the group consisting of amino acids, vitamin B, vitamin C, vitamin E, other vitamins, carotene, sphingosine, cadaverine and cobalamine.

52. The method of claim 45, wherein said test compound is selected from the group consisting of phospholipids, proteolipids, protein-nucleic acid complexes, protein RNA complexes, mitochondria, chloroplasts and other organelles.

53. A method of producing an isolated diverse population of F_{V}-like binding polypeptides, comprising coexpressing a first population of nucleic acids encoding a diverse population of V_{H}-like binding polypeptides and a second population of nucleic acids encoding a diverse population of V_{L}-like binding polypeptides, each of said V_{H}-like binding polypeptides comprising an unascertained combination of an immunoglobulin V_{H} region exon encoded polypeptide, a J_{H} region exon encoded polypeptide and a D region exon encoded polypeptide joined in a single polypeptide, or a functional fragment thereof, each of said V_{L}-like binding polypeptides comprising an unascertained combination of an immunoglobulin V_{L} region exon encoded polypeptide and a J_{L} region exon encoded polypeptide joined in a single polypeptide, or a functional fragment thereof, wherein said coexpressed first and second populations of binding polypeptides coassemble into unascertained V_{H}-like and V_{L}-like binding polypeptide combinations forming a diverse population of F_{V}-like binding polypeptides.
